(19) United States
(12) Patent Application Publication
(10) Pub. No.: US 2021/0349534 A1
(43) Pub. Date: Nov. 11, 2021

(54) EYE-TRACKING SYSTEM FOR ENTERING COMMANDS

(71) Applicant: Alcon Inc., Fribourg (CH)

(72) Inventor: Martin Eil, Berlin (DE)

(21) Appl. No.: 17/315,183

(22) Filed: May 7, 2021

Related U.S. Application Data
(60) Provisional application No. 63/021,231, filed on May 7, 2020.

(51) Int. Cl. G06F 3/01; G06F 3/0481; G06F 3/0482; G06T 19/00; G06F 3/033; H04N 13/332; A61B 90/00

(52) U.S. Cl. CPC G06F 3/013; G06F 3/04815; G06F 3/0482; G06T 19/006; A61B 2090/367; H04N 13/332; A61B 90/37; A61B 90/361; G06F 2203/04806; G06F 3/0334

(57) ABSTRACT

In certain embodiments, an eye-tracking system for entering commands includes a computer, a pair of three-dimensional glasses, and a display. The computer generates a three-dimensional graphical user interface with graphical elements, where each graphical element corresponds to a command. The pair of three-dimensional glasses directs the three-dimensional graphical user interface towards a pair of eyes of a user. The display displays the graphical elements to the user. The display includes light-emitting diodes configured to illuminate the pair of eyes and a camera configured to track movement of the pair of eyes relative to the three-dimensional graphical user interface to yield a pair of tracked eyes. The computer interprets a movement of the pair of tracked eyes relative to the three-dimensional graphical user interface as an interaction with a selected graphical element, and initiates the command corresponding to the selected graphical element.

### TECHNICAL FIELD

The present disclosure relates generally to controlling medical systems, and more specifically to a binocular system for entering commands.

### BACKGROUND

Medical devices can perform a wide variety of actions in response to commands from an operator. For example, an operator can select commands from a command panel to change magnification, focus, and brightness of a microscope. Entering commands for a medical device, however, has special concerns. Touching the command panel can contaminate the panel. Moreover, searching for the part of the panel to enter the command takes time and attention away from the user. Accordingly, known command panels are sometimes not suitable for certain situations.

### BRIEF SUMMARY

In certain embodiments, an eye-tracking system for entering commands includes a computer, a pair of three-dimensional (3D) glasses, and a display. The computer generates a 3D graphical user interface (GUI) with graphical elements, where each graphical element corresponds to a command. The pair of 3D glasses directs the 3D GUI towards a pair of eyes of a user. The display displays the graphical elements to the user. The display includes light-emitting diodes (LEDs) configured to create light reflections on the pair of eyes by illuminating the pair of eyes and a camera configured to track movement of the pair of eyes relative to the 3D GUI to yield a pair of tracked eyes. The computer interprets a movement of the pair of tracked eyes relative to the 3D GUI as an interaction with a selected graphical element, and initiates the command corresponding to the selected graphical element.

In certain embodiments, a method for entering commands using an eye-tracking system includes generating, by a computer, a three-dimensional (3D) graphical user interface (GUI) comprising one or more graphical elements. Each graphical element corresponds to a command. A display displays the 3D GUI and a pair of 3D glasses directs the 3D GUI comprising the one or more graphical elements toward a pair of eyes of a user. Two or more light-emitting diodes (LEDs) associated with the display illuminate the pair of eyes of the user. At least one camera associated with the display track a movement of the pair of eyes relative to the 3D GUI to yield a pair of tracked eyes. The pair of tracked eyes may be illuminated by the two or more LEDs. The computer interprets the movement of the pair of tracked eyes relative to the 3D GUI as an interaction with a selected graphical element and the computer initiates the command corresponding to the selected graphical element.

### BRIEF DESCRIPTION OF THE DRAWINGS

Embodiments of the present disclosure are described by way of example in greater detail with reference to the attached figures, in which:

FIG. 1 illustrates an embodiment of an eye-tracking system that allows a user to enter commands with eye movements;

FIG. 2 illustrates an embodiment of an eye-tracking system that includes a pair of 3D glasses; and

FIG. 3 illustrates an example of a method of entering commands with eye movements that may be used with the system of FIG. 1.

### DESCRIPTION OF EXAMPLE EMBODIMENTS

Referring now to the description and drawings, example embodiments of the disclosed apparatuses, systems, and methods are shown in detail. As apparent to a person of ordinary skill in the field, the disclosed embodiments are exemplary and not exhaustive of all possible embodiments.

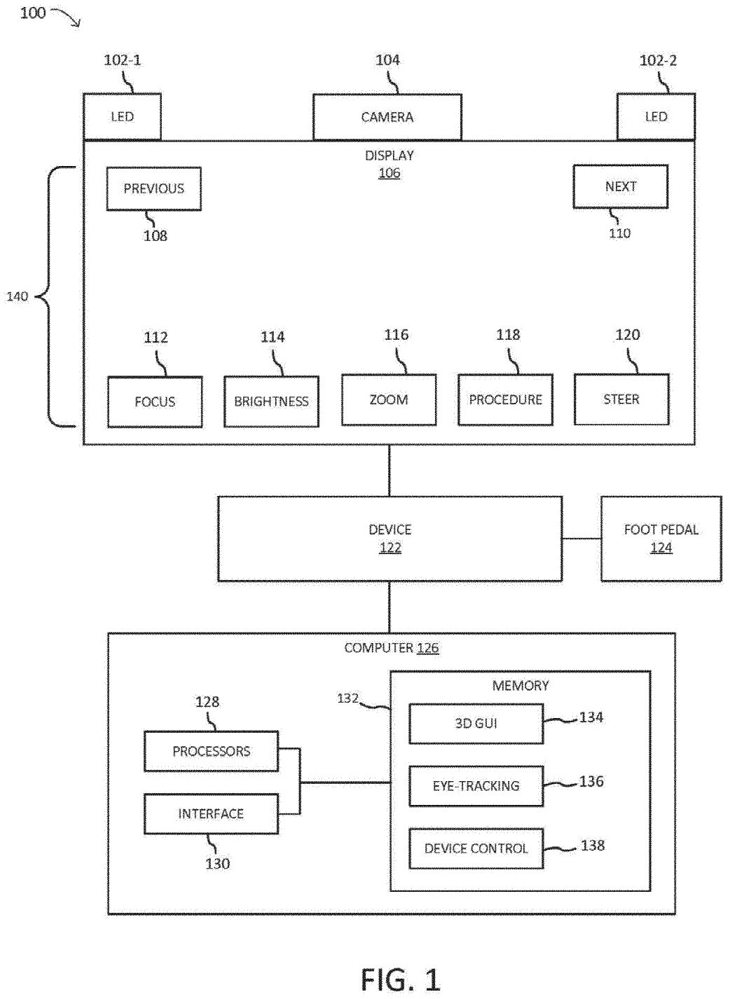

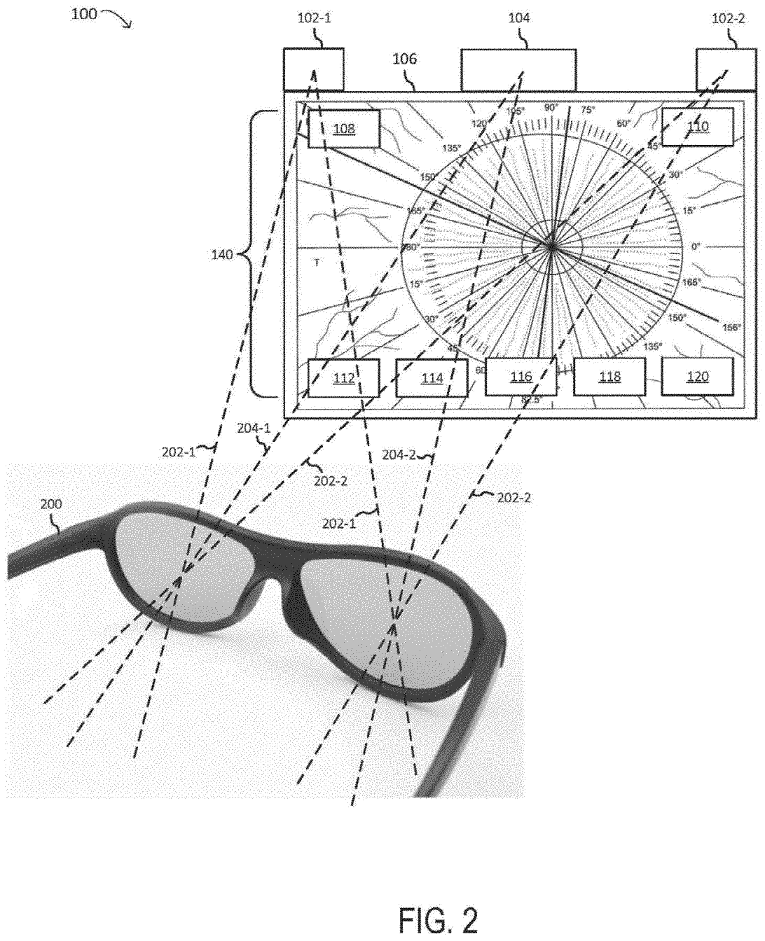

FIG. 1 illustrates an embodiment of an eye-tracking system 100 that allows a user to enter commands with eye movements. In the embodiment illustrated in FIG. 1, eye-tracking system 100 includes a computer 126, a display 106, and a foot pedal 124 communicatively coupled to a device 122. Computer 126 includes one or more processors 128, an interface 130, and one or more memories 132 that store logic such as computer programs for 3D graphical user interface (GUI) 134, eye-tracking 136, and device control 138. Display 106 includes light-emitting diodes (LEDs) 102-1 and 102-2 (collectively referred to herein as “LEDs 102”) and a camera 104. In addition, display 106 may display one or more graphical elements 140 of 3D GUI 134. In the embodiment illustrated in FIG. 2, graphical elements 140 include a focus element 112, a brightness element 114, a zoom element 116, a procedure element 118, and a steer element 120. Graphical elements 140 may additionally include a previous element 108 and a next element 110. In other embodiments, 3D GUI 134 may include additional, fewer, or any suitable combination of graphical elements 140 for allowing a user to enter commands with eye movements.

In an example of operation, eye-tracking system 100 allows a user to enter commands to any suitable device 122, e.g., such as a surgical camera. Computer 126 generates a 3D GUI 134 that includes one or more graphical elements 140. Each graphical element 140 corresponds to a command. Display 106 displays the 3D GUI 134 that includes the one or more graphical elements 140 such that at least one pair of 3D glasses may direct the 3D GUI 134 towards a pair of eyes of a user, e.g., a surgeon performing an ophthalmic procedure. Two or more LEDs 102 may be communicatively coupled to display 106 to illuminate the pair of eyes of the user. At least one camera 104 may be communicatively coupled to display 106 to track a movement of the pair of eyes relative to the 3D GUI 134, yielding a pair of tracked eyes. The pair of tracked eyes may be illuminated by the two or more LEDs 102. Computer 126 can interpret a movement of the pair of tracked eyes relative to the 3D GUI 134 as an interaction with a selected graphical element 140 and may initiate the command corresponding to the selected graphical element 140.

In one embodiment, device 122 may be a surgical camera with a resolution, image depth, clarity, and contrast that enables a high-quality image of patient anatomy. For example, a High Dynamic Range (HDR) surgical camera may be used to capture 3D images of an eye for performing actions during surgical procedures, e.g., ophthalmic procedures. Device 122 may be communicatively coupled with display 106 (e.g. via a wired connection, a wireless connection, etc.) and the display 106 can display a stereoscopic representation of the 3D image providing a surgeon, staff, students, and/or other observers depth perception into the eye anatomy. Device 122 can also be used to increase magnification of the eye anatomy while maintaining a wide field of view. The stereoscopic representation of the 3D image can be viewed on display 106 with 3D glasses. With the stereoscopic representation of the 3D image displayed on the display 106, a user can perform surgical procedures on a patient's eye while in a comfortable position without bending over a microscope eyepiece and straining the neck.

In certain embodiments, computer 126 generates a 3D GUI 134, which is directed toward a pair of eyes of a user via display 106. The 3D GUI 134 includes one or more graphical elements 140, which may have any suitable size or shape. Each graphical element 140 corresponds to a command to device 122, typically to perform an action, e.g., accept a selection or setting defined by the user, perform a user-selected operation programmed into computer 126, display information requested by the user, or other suitable action. In the embodiment illustrated in FIG. 1, graphical elements 140 include a previous element 108, a next element 110, a focus element 112, a brightness element 114, a zoom element 116, a procedure element 118, and a steer element 120. Previous element 108 corresponds to a command to move backwards, e.g., move to a previous menu, to a previous option on a list of the menu, and/or to a previous step in a surgical procedure. Next element 110 corresponds to a command to move forwards, e.g., move to a next menu, to a next option on a list of the menu, and/or to a next step in a surgical procedure. Focus element 112 corresponds to a command to control a focus of one or more 3D images of a surgical procedure captured by device 122. Brightness element 114 corresponds to a command to control a brightness level of the one or more 3D images of the surgical procedure, e.g., an amount of light received through a lens of device 122. Zoom element 116 corresponds to a command to control an angle of view of the one or more 3D images of the surgical procedure. Procedure element 118 corresponds to a command to display on display 106 a sequence of steps comprising a procedure paradigm associated with the surgical procedure. Steer element 120 corresponds to a command to control a movement of device 122, e.g., along x, y, and/or z axes during the surgical procedure. A user may enter a command by making his/her gaze interact with the graphical element 140 corresponding to the command displayed on display 106.

In one embodiment, computer 126 may interpret an interaction as a movement of the eye (e.g., moving or directing eye gaze or blinking the eye) relative to a graphical element 140 that indicates, e.g., selection of the graphical element 140. In one embodiment, the user may direct his/her gaze at the graphical element 140 for at least a predefined number of seconds, e.g., at least 3, 5, or 10 seconds such that the predefined number of seconds indicates a selection of a selected graphical element 140. In another embodiment, the user may direct his/her gaze at the graphical element 140 and may blink a predetermined number of times, e.g., 1, 2, or 3 times to indicate a selection of a selected graphical element 140. In other embodiments, the interaction may be confirmed by movement of another part of the user's body. For example, the user may direct his/her gaze towards a graphical element 140 to select the graphical element 140, and then confirm selection of the graphical element 140 by actuating foot pedal 124 with his/her foot or pressing a physical button with his/her hand. In the embodiment illustrated in FIG. 1, foot pedal 124 may be communicatively coupled to display 106 via device 122. In another embodiment, foot pedal 124 may be directly communicatively coupled to display 106. In certain embodiments, 3D GUI 134 can indicate if a user's gaze has interacted with or selected a graphical element 140. For example, 3D GUI 134 can highlight (e.g., make brighter or change color of) a graphical element 140 displayed on display 106 that the user's gaze has selected. The user may confirm selection by, e.g., blinking, moving a hand or foot, and/or actuating foot pedal 124.

In one embodiment, eye-tracking program 136 of computer 126 interprets a movement of the pair of tracked eyes relative to 3D GUI 134 as an interaction with a selected graphical element 140, and device control program 138 initiates the command corresponding to the selected graphical element 140. Eye-tracking program 136 includes known algorithms to determine a gaze direction of the eye from image data generated by camera 104. Processors 128 perform calculations based on the algorithms to determine the gaze direction. Additionally, eye-tracking programs 136 can detect other movement of the eye, e.g., such as a blink. Given the gaze direction and position of 3D GUI 134, processors 128 can determine if the gaze has interacted with a graphical element 140 of 3D GUI 134 in a manner that indicates selection of the graphical element 140. If a graphical element 140 is selected, device control program 138 initiates the command corresponding to the selected element.

In one embodiment, display 106 can display a stereoscopic representation of one or more 3D images of a surgical procedure captured by device 122. Display 106 can additionally display 3D GUI 134 such that 3D GUI 134 may be superimposed over the one or more 3D images of the surgical procedure displayed to the user. In one embodiment, display 106 can receive information (e.g., surgical parameters) from device 122 and can display the information along with the stereoscopic representation of the one or more 3D images. In another embodiment, display 106 may also receive signals from device 122 for performing operations (e.g., starting and stopping video recording). In one embodiment, display 106 may be or include a 3D monitor used to display the stereoscopic representation of the one or more 3D images of a surgical procedure. In the embodiment illustrated in FIG. 1, display 106 may include LEDs 102 and camera 104.

In one embodiment, LEDs 102 may illuminate a pair of tracked eyes during a surgical procedure. Specifically, LEDs 102 can illuminate the pair of tracked eyes to create light reflections that can be detected by camera 104 to generate image data. LEDs 102 may illuminate with any suitable light, e.g., visible and/or infrared (IR) light. In one embodiment, LEDs 102 may be or include solid state lighting (SSL) devices that emit light in the IR range of the electromagnetic radiation spectrum, e.g., 700 nanometers (nm) to 1 millimeter (mm) range. When used with an infrared camera, IR LEDs 102 can illuminate the pair of tracked eyes while remaining invisible to the naked eye. In this way, IR LEDs 102 may illuminate the pair of tracked eyes without causing a visual distraction, e.g., such as bright lights emitted into the pair of tracked eyes of the user during the surgical procedure. Although LEDs 102 are positioned above display 106 in the embodiment illustrated in FIG. 1, LEDs 102 may be positioned in any suitable location to track movement of the pair of tracked eyes. Additionally, any suitable number of LEDs 102 may be used to track movement of the pair of tracked eyes. In other embodiments, any suitable illuminator may be used, e.g., such as a halogen lamp, infrared lamp, filtered incandescent lamp, and the like.

In one embodiment, camera 104 may track movement of a pair of tracked eyes relative to graphical elements 140 of 3D GUI 134 displayed on display 106 during a surgical procedure. Specifically, camera 104 may detect light reflections from the pair of tracked eyes illuminated by LEDs 102, e.g., from the cornea (anterior surface), pupil center, limbus, lens (posterior surface), and/or any other suitable part of the pair of tracked eyes. Camera 104 may generate image data describing the pair of tracked eyes and can send the image data to computer 126. In particular, camera 104 may generate image data describing the light reflections from the pair of tracked eyes and can transmit the image data (e.g. via a wired connection, a wireless connection, etc.) to eye-tracking program 136 of computer 126. In response to receiving the image data, eye-tracking program 136 may use the image data to interpret a movement of the pair of tracked eyes relative to the 3D GUI 134 as an interaction with a selected graphical element 140. Similarly, device control program 138 of computer 126 may use the image data generated by camera 104 to initiate the command corresponding to the selected graphical element 140. Although camera 104 is positioned above display 106 in the embodiment illustrated in FIG. 1, camera 104 may be positioned in any suitable location to track movement of the pair of tracked eyes. Additionally, any suitable number of cameras 104 may be used to track movement of the pair of tracked eyes. In other embodiments, any suitable camera may be used, e.g., such as a thermographic camera, a short wavelength infrared camera, a mid-wavelength infrared camera, a long wavelength infrared camera, and the like.

FIG. 2 illustrates an embodiment of an eye-tracking system 100 that includes a pair of 3D glasses 200. In the embodiment illustrated in FIG. 2, 3D glasses 200 can direct 3D GUI 134 towards a pair of eyes of a user, e.g., a surgeon performing an ophthalmic procedure. LEDs 102 can illuminate the pair of eyes of the user by emitting light beams, e.g., light beams 202-1 and 202-2 (collectively referred to herein as “light beams 202”). This is shown in FIG. 2 where LED 102-1 emits light beams 202-1 and LED 102-2 emits light beams 202-2. Each light beam 202 emitted by LEDs 102 may travel through a lens of 3D glasses 200 to generate light reflections 204-1 and 204-2 (collectively referred to herein as “light reflections 204”) from the pair of eyes. Light reflections 204 from the pair of eyes may be tracked by camera 104, yielding a pair of tracked eyes. For example, light beams 202 may cause light reflections 204 from the corneas of the pair of tracked eyes that camera 104 may use to track a movement of the pair of tracked eyes relative to 3D GUI 134. The pair of tracked eyes of the user may be continuously illuminated by light beams 202 emitted from LEDs 102 throughout the surgical procedure such that camera 104 may track movements of the pair of tracked eyes based on light reflections 204. Movements of the pair of tracked eyes relative to 3D GUI 134 may be interpreted by computer 126 (not shown in figure) as an interaction with a selected graphical element 140 and computer 126 may initiate the command corresponding to the selected graphical element 140. For example, camera 104 may track light reflections 204 to generate image data describing the pair of tracked eyes. Computer 126 may interpret the image data to determine that the pair of tracked eyes initiated an interaction (e.g., a gaze) with focus element 112. Upon interpreting the movement of the pair of tracked eyes as an interaction with focus element 112 and/or receiving a predefined number of blinks, computer 126 may initiate a focus command instructing device 122 to control the focus of one or more 3D images of a surgical procedure captured by device 122. In one embodiment, 3D glasses 200 may include one or more sensors (not shown in figure) disposed within 3D glasses 200 such that the one or more sensors can track the movement of the pair of tracked eyes relative to 3D GUI 134. For example, LEDs 102 may illuminate the pair of tracked eyes and the one or more sensors may determine if the pair of tracked eyes initiated an interaction with a selected graphical element 140.

In some embodiments, a position of the head of the user in relation to display 106 may be determined to calibrate eye-tracking system 100 prior to a surgical procedure. In one embodiment, a user may calibrate camera 104 such that camera 104 can accurately generate image data describing a pair of tracked eyes. For example, display 106 may display a prompt instructing the user to look at a specific graphical element 140 displayed on display 106 while the user is in a seated position typically used during a surgical procedure. Computer 126 may associate a trajectory of the pair of tracked eyes of the user in the seated position with the location of the specific graphical element displayed on display 106 to calibrate eye-tracking system 100. In another embodiment, eye-tracking system 100 may initiate a calibration process without receiving image data from a user. For example, eye-tracking system 100 may employ a built-in self test (BIST) upon system initialization used to calibrate camera 104 in relation to the surrounding environment.

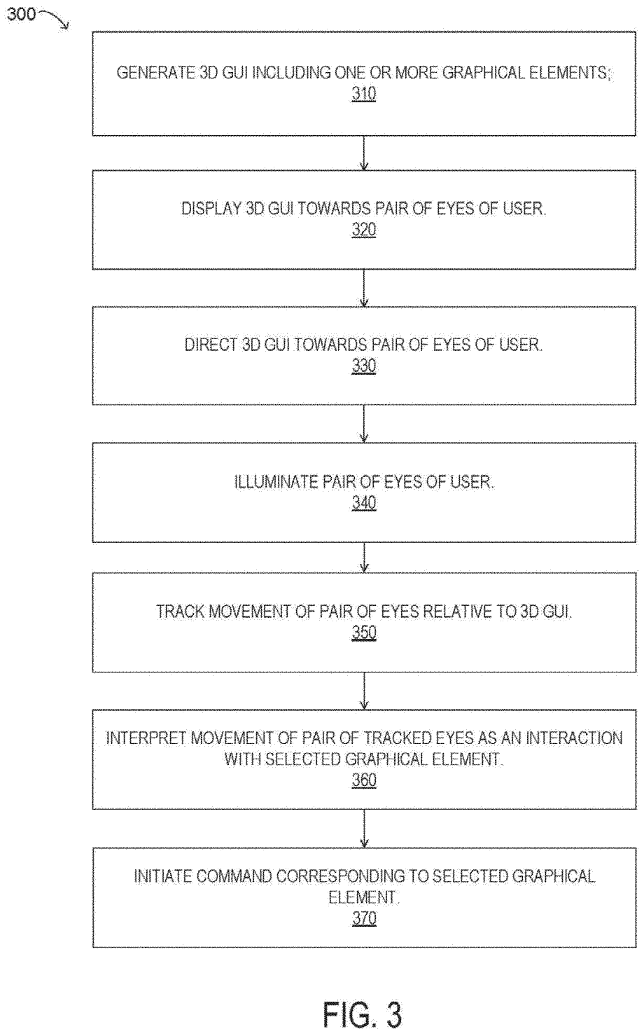

FIG. 3 illustrates an example of a method of entering commands with eye movements that may be used with system 100 of FIGS. 1 and 2. The method starts at step 310, where computer 126 generates a three-dimensional (3D) graphical user interface (GUI) 134 that includes one or more graphical elements 140. Each graphical element 140 corresponds to a command. At step 320, a display 106 displays the 3D GUI 134 that includes the graphical elements 140. A pair of 3D glasses 200 directs the 3D GUI 134 towards a pair of eyes of a user at step 330. At step 340, two or more light-emitting diodes (LEDs) 102 illuminate the pair of eyes. The two or more LEDs 102 may be associated with display 106. For example, LEDs 102 may be communicatively coupled to display 106 as illustrated in FIG. 2. At step 350, a camera 104 may track a movement of the pair of eyes relative to the 3D GUI to yield a pair of tracked eyes. The pair of tracked eyes may be illuminated by the two or more LEDs 102. The computer 126 interprets the movement of the pair of tracked eyes relative to the 3D GUI as an interaction with a selected graphical element 140 at step 360. At step 370, the computer 126 initiates the command that corresponds to the selected graphical element.

A component (e.g., a computer) of the systems and apparatuses disclosed herein may include an interface, logic, and/or memory, any of which may include hardware and/or software. An interface can receive input to the component, provide output from the component, and/or process the input and/or output. Logic can perform the operations of the component, e.g., execute instructions to generate output from input. Logic may be a processor, such as one or more computers or one or more microprocessors (e.g., a chip that resides in computers). Logic may be computer-executable instructions encoded in memory that can be executed by a computer, such as a computer program or software. A memory can store information and may comprise one or more tangible, non-transitory, computer-readable, computer-executable storage media. Examples of memory include computer memory (e.g., Random Access Memory (RAM) or Read Only Memory (ROM)), mass storage media (e.g., a hard disk), removable storage media (e.g., a Compact Disk (CD) or a Digital Video Disk (DVD)), and network storage (e.g., a server or database).

Although this disclosure has been described in terms of certain embodiments, modifications (such as substitutions, additions, alterations, or omissions) of the embodiments will be apparent to those skilled in the art. Accordingly, modifications may be made to the embodiments without departing from the scope of the invention. For example, modifications may be made to the systems and apparatuses disclosed herein. The components of the systems and apparatuses may be integrated or separated, and the operations of the systems and apparatuses may be performed by more, fewer, or other components. As another example, modifications may be made to the methods disclosed herein. The methods may include more, fewer, or other steps, and the steps may be performed in any suitable order.

What is claimed is:

1. An eye-tracking system for entering commands, the system comprising:
a computer configured to generate a three-dimensional (3D) graphical user interface (GUI) comprising one or more graphical elements, each graphical element corresponding to a command;
at least one pair of 3D glasses configured to direct the 3D GUI comprising the one or more graphical elements towards a pair of eyes of a user;
a display configured to display the one or more graphical elements to the user, the display including:
two or more light-emitting diodes (LEDs) configured to illuminate the pair of eyes; and
at least one camera configured to track movement of the pair of eyes relative to the 3D GUI to yield a pair of tracked eyes, the pair of tracked eyes illuminated by the two or more LEDs;
the computer further configured to:
interpret a movement of the pair of tracked eyes relative to the 3D GUI as an interaction with a selected graphical element; and
initiate the command corresponding to the selected graphical element.

2. The eye-tracking system of claim 1, further comprising:
a device configured to capture one or more 3D images of a surgical procedure, the device communicatively coupled to the display, the display configured to display the one or more 3D images of the surgical procedure and the one or more graphical elements to the user.

3. The eye-tracking system of claim 2, wherein the one or more graphical elements are superimposed over the one or more 3D images of the surgical procedure displayed to the user on the display.

4. The eye-tracking system of claim 2, wherein the one or more graphical elements comprise at least one of the following:
a focus element corresponding to a command to control a focus of the one or more 3D images of the surgical procedure;
a brightness element corresponding to a command to control a brightness level of the one or more 3D images of the surgical procedure;
a zoom element corresponding to a command to control an angle of view of the one or more 3D images of the surgical procedure;
a procedure element corresponding to a command to display on the display a sequence of steps comprising a procedure paradigm associated with the surgical procedure;
a steer element corresponding to a command to control a movement of the device;

5. The eye-tracking system of claim 1, wherein the one or more graphical elements comprise at least one of the following:
a previous element corresponding to a command to move backwards; and
a next element corresponding to a command to move forwards.

6. The eye-tracking system of claim 1, wherein the two or more LEDs are comprised of infrared (IR) LEDs.

7. The eye-tracking system of claim 1, wherein the interaction with the selected graphical element comprises a predefined number of blinks generated by the pair of tracked eyes of the user, the predefined number of blinks indicating a selection of the selected graphical element.

8. The eye-tracking system of claim 1, wherein the interaction with the selected graphical element comprises a predefined number of seconds in which the pair of tracked eyes of the user interacts with the selected graphical element, the predefined number of seconds indicating a selection of the selected graphical element.

9. The eye-tracking system of claim 1, wherein the interaction with the selected graphical element comprises a user confirmation of the selected graphical element via a foot pedal, the foot pedal communicatively coupled to the display.

10. The eye-tracking system of claim 1, further comprising:
one or more sensors disposed within the at least one pair of 3D glasses, the one or more sensors configured to track the movement of the pair of tracked eyes relative to the 3D GUI.

11. A method for entering commands using an eye-tracking system, comprising:
generating, by a computer, a three-dimensional (3D) graphical user interface (GUI) comprising one or more graphical elements, each graphical element corresponding to a command;
displaying, by a display, the 3D GUI comprising the one or more graphical elements;
directing, by at least one pair of 3D glasses, the 3D GUI comprising the one or more graphical elements towards a pair of eyes of the user;
illuminating, by two or more light-emitting diodes (LEDs) associated with the display, the pair of eyes of the user;
tracking, by at least one camera associated with the display, a movement of the pair of eyes relative to the 3D GUI to yield a pair of tracked eyes, the pair of tracked eyes illuminated by the two or more LEDs;
interpreting a movement of the pair of tracked eyes relative to the 3D GUI as an interaction with a selected graphical element; and
initiating the command corresponding to the selected graphical element.

12. The method of claim 11, further comprising:
capturing, by a device, one or more 3D images of a surgical procedure, the device communicatively coupled to the display, the display configured to display the one or more 3D images of the surgical procedure and the one or more graphical elements to the user.

13. The method of claim 12, wherein the one or more graphical elements are superimposed over the one or more 3D images of the surgical procedure displayed to the user on the display.

14. The method of claim 12, wherein the one or more graphical elements comprise at least one of the following:
a focus element corresponding to a command to control a focus of the one or more 3D images of the surgical procedure;
a brightness element corresponding to a command to control a brightness level of the one or more 3D images of the surgical procedure;
a zoom element corresponding to a command to control an angle of view of the one or more 3D images of the surgical procedure;
a procedure element corresponding to a command to display on the display a sequence of steps comprising a procedure paradigm associated with the surgical procedure;
a steer element corresponding to a command to control a movement of the device;

15. The method of claim 11, wherein the one or more graphical elements comprise at least one of the following:
a previous element corresponding to a command to move backwards; and
a next element corresponding to a command to move forwards.

16. The method of claim 11, wherein the two or more LEDs are comprised of infrared (IR) LEDs.

17. The method of claim 11, wherein the interaction with the selected graphical element comprises a predefined number of blinks generated by the pair of tracked eyes of the user, the predefined number of blinks indicating a selection of the selected graphical element.

18. The method of claim 11, wherein the interaction with the selected graphical element comprises a predefined number of seconds in which the pair of tracked eyes of the user interacts with the selected graphical element, the predefined number of seconds indicating a selection of the selected graphical element.

19. The method of claim 11, wherein the interaction with the selected graphical element comprises a user confirmation of the selected graphical element via a foot pedal, the foot pedal communicatively coupled to the display.

20. The method of claim 11, further comprising:
one or more sensors disposed within the at least one pair of 3D glasses, the one or more sensors configured to track the movement of the pair of tracked eyes relative to the 3D GUI.
